(19) United States
(12) Patent Application Publication
(10) Pub. No.: US 2024/0099646 A1
(43) Pub. Date: Mar. 28, 2024

(54) DIAGNOSTIC DEVICE AND SYSTEM AND METHOD FOR THE PRODUCTION THEREOF

(71) Applicants: MEDICA S.P.A., Medolla (IT); SCUOLA SUPERIORE DI STUDI UNIVERSITARI E DI PERFEZIONAMENTO SANT'ANNA, Pisa (IT)

(72) Inventors: Marcello Chiurazzi, Pisa (IT); Giorgia Spraeafico, Pisa (IT); Andrea Firrincieli, Pisa (IT); Gastone Ciuti, Piasa (IT); Cesare Stefanini, Pisa (IT); Francesco Paolo Falotico, Pisa (IT); Davide Bagnoli, Medolla (IT); Sergio Emiliani, Medolla (IT)

(21) Appl. No.: 18/275,629

(22) PCT Filed: Feb. 11, 2022
(86) PCT No.: PCT/IB2022/051248

§ 371 Date: Aug. 3, 2023

(30) Foreign Application Priority Data
Feb. 12, 2021 (IT) 102021000003197

(51) Int. Cl. A61B 5/00

(52) U.S. Cl. CPC A61B 5/4233; A61B 5/6847; A61B 2562/0214; A61B 2562/12; A61B 2562/164; A61B 2562/182

(57) ABSTRACT

A method for the production of a diagnostic device comprising the steps of: obtaining a blank from a plastic material sheet; the blank comprising one or more support bands provided with electrical tracks; providing one or more sensitive elements, in particular detection pads, for each band, being configured to generate signals, in particular pressure signals and being connected to the electrical tracks for the transmission of signals; applying a first electrically conductive coating on the blank in the area of the sensitive elements; creating a main structure from the blank, in particular by rolling the blank, keeping the first electrically conductive coating radially on the inside of the main structure; and creating an auxiliary matrix which wraps the main structure and fills the gaps between the support bands, so as to obtain a tubular body.

### CROSS-REFERENCE TO RELATED APPLICATIONS

This patent application claims priority from Italian patent application no. 102021000003197 filed on Feb. 12, 2021, the entire disclosure of which is incorporated herein by reference.

### TECHNICAL FIELD

The present invention relates to a diagnostic device and system and a method for the production thereof.

In particular, the present invention relates to a diagnostic device for performing endoscopic clinical examinations. More specifically, the present invention relates to a diagnostic device for carrying out clinical examinations of the esophagus, stomach, intestine or similar. For example, it is also possible to use the diagnostic device according to the present invention to perform different types of clinical examinations, such as cardiac examinations.

Advantageously, the present invention finds application in the diagnostic procedure of esophageal manometry.

### PRIOR ART

The use of endoscopic diagnostic devices in clinical examinations is known, which are introduced into a cavity (esophagus, stomach, intestine, uterus, circulatory system) of the human body through natural orifices (nose, mouth, rectum and similar) or through artificial orifices (namely, passageways created by incision of the human body to access certain cavities) to acquire the information required for the respective diagnostic examination to be performed. Once the diagnostic device has been inserted into the patient's body through the orifice, the diagnostic device must follow a path inside the cavity which can be particularly tortuous.

For example, for an esophageal manometry, the diagnostic device must follow a path that has several bends which may require the diagnostic device to be bent at an angle of even more than 30°.

To carry out the esophageal manometry there are two types of diagnostic devices, known as: water-perfused catheter and solid-state catheter.

The water-perfused catheter diagnostic device has too low a dynamic response (refresh rate of the order of 20 Hz) and provides accurate data. Therefore, this type of instrumentation is almost no longer used, solid-state diagnostic devices being preferred. The solid-state catheters currently known have a solid circular section, with a rigid structure having a rectangular section inside [a thin/laminar body with a dimension (length) predominant over the others] positioned in the area of the central axis to provide axial rigidity to the system.

Given the shape of the diagnostic device, it can only be bent in one direction, which is substantially perpendicular to a longitudinal axis.

However, the bending required for the correct insertion of the catheter into the cavity may take place on different anatomical planes. Therefore, during insertion of a solid-state catheter of a known type, it is necessary to carry out twisting manoeuvres which are difficult for the patient to tolerate.

Furthermore, a known type of solid-state catheter has the disadvantage that its solid section does not allow the use of endoscopic guide, which would be necessary for patients with abnormalities that may obstruct the passage of the diagnostic device. Therefore, it is often not possible to perform the examination with the known type of diagnostic devices in patients with severe abnormalities.

Furthermore, the known type of solid-state catheter is expensive to manufacture and is multiuse, namely, it must be washed and sterilised after each use so that it can be used for another patient later on. Given that the known type of solid-state catheter is multiuse, it must guarantee a prolonged use over time without presenting any deterioration resulting in alteration of the examination performed. Therefore, the known solid-state catheter must be produced with a material that is easy to clean, resistant over time both to corrosion (due, for example, to gastric juices and to cleaning/sterilisation products and methods) and in terms of fatigue strength (phenomenon due to the application of one-way loads, namely applied in one direction with two opposite ways, variable over time and linked to the succession of elastic bends).

In other words, the material used must be easy to clean, resistant and must be able to deform elastically (in the same direction, but in two opposite ways), without presenting any residual deformation after use. The materials typically used are therefore very expensive and require particular processing, which makes the diagnostic device itself difficult to produce and very expensive. Therefore, to amortise the high cost of the diagnostic device, it is necessary to use it for a number of procedures ranging from 100 to 200 times.

In addition, the known type of solid-state catheter is often damaged due to the high and repeated number of uses and stresses to which it is subjected. Therefore, the known type of solid-state catheter often requires extraordinary maintenance, further increasing its management cost.

### DESCRIPTION OF THE INVENTION

Therefore, the aim of the present invention is to provide a diagnostic device and a method for the production thereof which are free from the drawbacks of the state of the art and which are easy and inexpensive to implement.

According to the present invention, a method for the production of the diagnostic device is provided according to what is claimed in the appended claims.

According to the present invention, a diagnostic device is provided according to what is claimed in the appended claims.

According to the present invention, a diagnostic system is provided according to what is claimed in the appended claims.

### BRIEF DESCRIPTION OF THE DRAWINGS

For a better understanding of the invention embodiments are described, purely by way of example, where:

FIG. 1 is a schematic view of a possible application of a diagnostic device object of the present invention;

FIG. 2 is a schematic view of the diagnostic device object of the present invention according to a first and preferred embodiment;

FIG. 3a is a schematic and perspective view of a detail of the diagnostic device of FIG. 2;

FIG. 3b illustrates a detail on an enlarged scale of the detail of FIG. 3a;

FIG. 3c illustrates a cross-section of the detail of FIG. 3a;

FIGS. 4a, 4b and 4c are similar to FIGS. 3a, 3b and 3c respectively and illustrate a variation of the detail of FIG. 3;

FIGS. 5a, 5b and 5c are similar to FIGS. 3a, 3b and 3c respectively and illustrate a further variation of the detail of FIG. 3;

FIG. 6 is a schematic and perspective view of a blank for manufacturing the diagnostic device illustrated in FIG. 2;

FIGS. 7a-7d are schematic views of respective steps for manufacturing the diagnostic device;

FIG. 8 is a block diagram that illustrates the operation of the diagnostic device; and

FIGS. 9a and 9b are cross-sections of the diagnostic device along the line IX-IX of FIG. 2.

### PREFERRED EMBODIMENTS OF THE INVENTION

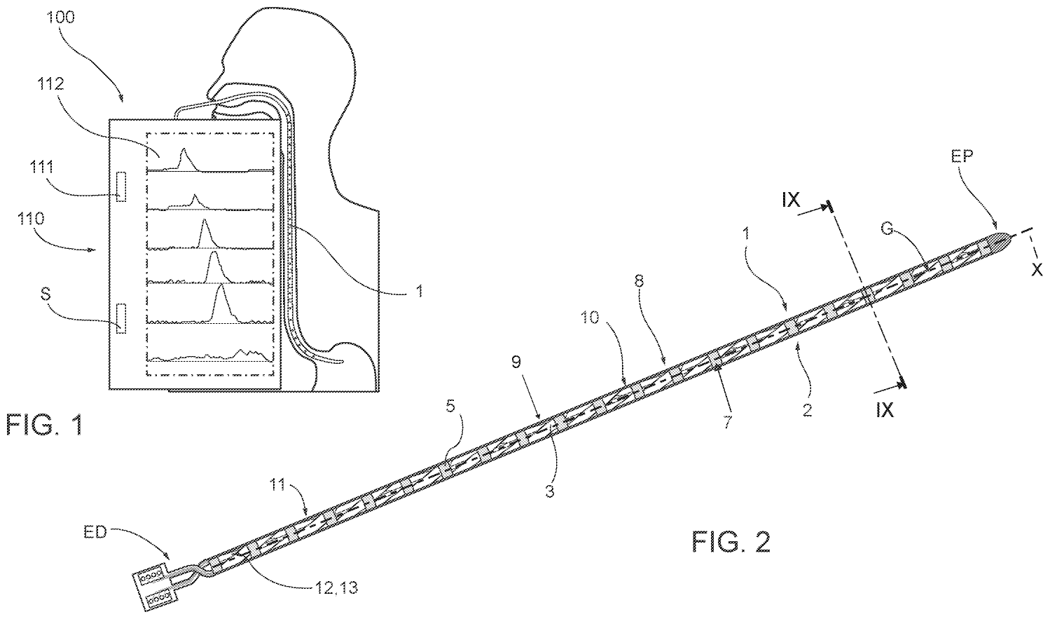

In FIG. 1 the number 1 indicates, as a whole, a diagnostic device according to the present invention during use. The diagnostic device 1 is configured to be introduced into a cavity of the human body through an orifice, as will be shown in more detail below.

By way of example, without loss of generality, FIG. 1 illustrates a diagnostic device 1 according to the present invention during a high-resolution esophageal manometry (also referred to simply with the acronym HRM—High Resolution Manometry).

The esophageal manometry is performed by means of a system 100 that comprises, in turn, a diagnostic device 1 (substantially similar to a catheter, namely, a probe) provided with pressure sensors 5 (hereinafter called indistinctly detection pads or sensitive elements 5) distributed along its own central axis X, as will be described in detail hereinafter, and with a control unit 110. The diagnostic device 1 is configured to exchange signals detected by the pressure sensors 5 with the control unit 110. The exchange of signals between the diagnostic device 1 and the control unit 110 may take place via cables or wirelessly. Purely by way of example, in the figures the connection between the diagnostic device 1 and the control unit 110 for the exchange of signals is physical, namely, by means of a cable.

The control unit 110 comprises, in turn: a software component S for detecting and processing signals; and/or a memory unit 111; and a user interface 112 for exchanging input and/or output data with an external user. According to the example illustrated, the user interface 112 is a display that shows, at output, graphs relative to the variation in pressure along the central axis X of the diagnostic device 1 during use.

According to the example illustrated in FIG. 1, the user interface 112 may be integrated in the body of the control unit 110. According to a variation not illustrated and without loss of generality, the user interface 112 may be part of a physical component divided from the control unit 110 and stand-alone (for example, a remote unit, a mobile device such as a Smartphone or iPad or similar), in this case the user interface 112 is configured to exchange input and output data with the software component S of the control unit 110.

Advantageously, the system 100, in particular the software component S, is configured to acquire and analyse the signals relative to the peristaltic pressure wave that travels down the esophagus during swallowing.

In this regard, the diagnostic device 1 is configured to detect signals, which once processed by the software component S are useful to discriminate, during swallowing, a physiological behaviour from a pathological behaviour.

Advantageously, the presence of the user interface 112 allows information to be exchanged directly and substantially intuitively with an operator to show the patient swallowing.

According to what is illustrated in FIG. 1, the diagnostic device 1 is an at least partially flexible body that is inserted through the nasal cavity into the esophagus and is pushed up to the opening of the stomach.

What follows in relation to the diagnostic device 1 may be applied, without difficulty, to any diagnostic device that has a shape similar to a catheter.

In use, the diagnostic device 1, after being positioned inside the esophagus as indicated above, is capable of acquiring, as will be better illustrated below, a plurality of signals relative to intra-esophageal pressure in the various segments of the esophagus (as illustrated in the pressure-time diagram shown on the left side of FIG. 1). Normally, the pressure analysis is carried out during swallowing of small sips of water by the patient who is in the supine position, therefore in the diagrams shown by the user interface 112 the pressure wave moves along the diagnostic device 1 as a function of swallowing of the sip of water by the patient.

According to what is illustrated in FIG. 2, the diagnostic device 1 has a distal end ED and a proximal end EP. The distal end ED is configured to be connected to the control unit 110, preferably via a physical connection, for example, electrical cables. Alternatively, the diagnostic device 1 may be connected to the control unit 110 via a wireless connection. On the other hand, the proximal end EP is the end that is inserted through the orifice into the patient's cavity (according to the example illustrated, through the nasal cavity into the patient's esophagus).

Advantageously, the diagnostic device 1 comprises a main internal structure 2 that has a development predominant along the central axis X and having a tubular shape, in particular having an annular section.

Advantageously, the diagnostic device 1 has the central axis X which is also an axis of symmetry. In other words, the diagnostic device 1 has a tubular body 9 that is substantially axisymmetric relative to the central axis X.

The diagnostic device 1 is flexible. The diagnostic device 1, in particular its tubular body 9, is configured to selectively be arranged in any position between a linear configuration (illustrated for example in FIGS. 2-5), where the tubular body 9 is substantially straight, and a bent configuration (illustrated for example in FIG. 1), wherein the tubular body 9 is flexed (namely, bent), around one or more axes, so as to adapt to the path to be followed in the patient's body.

Advantageously, the tubular body 9 of the diagnostic device 1 is elastically deformable along any deformation direction that is transverse, in particular orthogonal, to the longitudinal direction (parallel to the central axis X in the linear configuration) of the main structure 2 and the extent of deformation is substantially the same for each deformation direction. In other words, the main structure 2 may be bent (namely, is bendable) relative to a plurality of deformation axes (in particular, radial axes) with different directions from each other. Therefore, the diagnostic device 1 is bendable around any deformation axis and does not have a preferred deformation direction (namely, bending direction).

The main structure 2 is the internal skeleton of the diagnostic device 1.

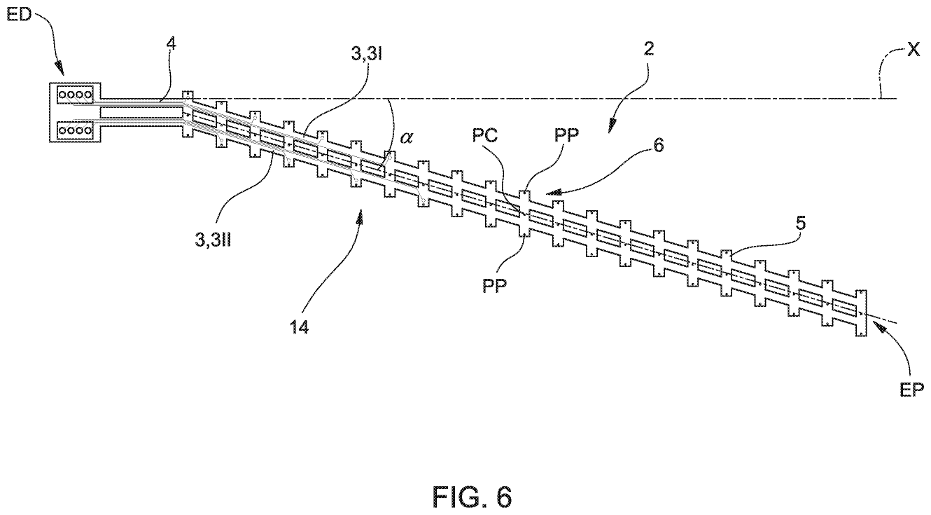

The main structure 2 comprises at least two support bands 3 each of which is provided with: at least one or more electrical tracks 4 (illustrated by way of example schematically and partially in FIG. 6); and one or more detection pads 5. Each electrical track 4 terminates on a different detection pad 5. Preferably, each support band 3 has a plurality of detection pads 5 interspersed from one another along the central axis X. The detection pads 5 are sensitive elements which are configured to generate signals, in particular pressure signals.

Advantageously, each detection pad 5 is obtained by a local variation of surface of the respective support band 3.

Advantageously, both support bands 3 are manufactured from a same sheet of insulating material, namely, a material that does not conduct electric current. The detection pads 5 define the detection sensors to acquire a respective signal, such as, for example, intra-esophageal pressure.

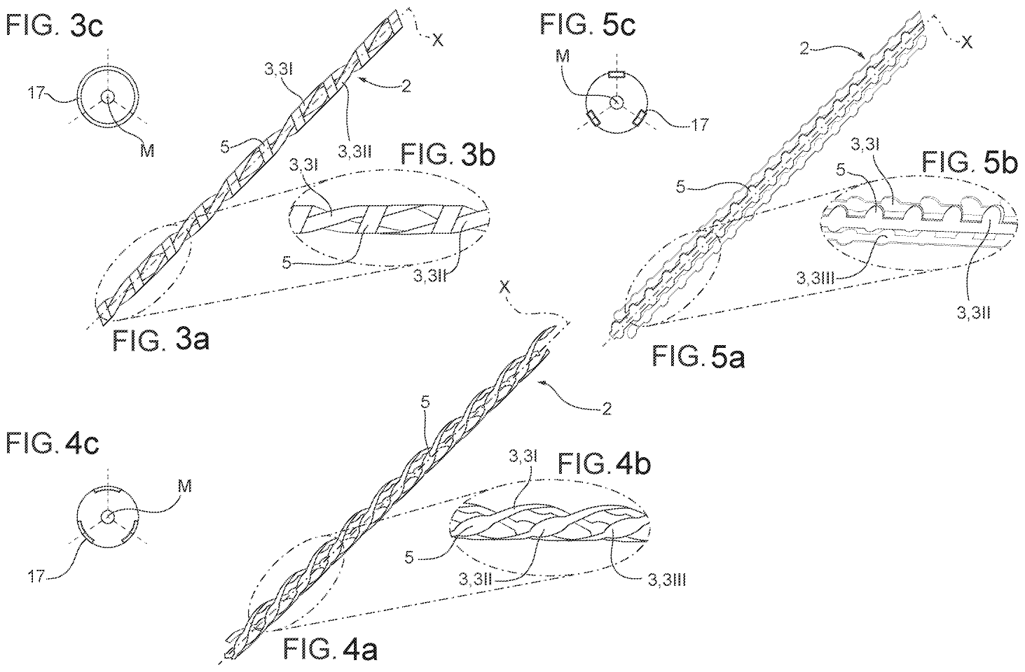

As can be seen in FIGS. 3-5, the detection pads 5 are spaced apart, in particular equidistant, from one another. In particular, the detection pads 5 are spaced apart by 10 mm, as defined by the Chicago classification.

According to a first and preferred embodiment, illustrated in FIGS. 2 and 3, there are two support bands 3, identified hereinafter and in the Figures with 31 and 311. Each support band 31 or 311 extends helically, namely is screw wound, along the central axis X defining the main structure 2. In other words, the main structure 2 comprises the support bands 31, 311 arranged helically as will be described in detail hereinafter. The support bands 31 and 311 and the detection pads 5 interspersed from one another give the main structure 2 a netlike shape.

Advantageously, according to what is illustrated in FIGS. 2 to 6, each detection pad 5 is obtained directly on each support band 31 or 311 with no interruption.

Advantageously, according to the embodiment illustrated in FIGS. 3 and 6, each detection pad 5 is annular and is substantially a partition 6 transverse to the central axis X and which is interposed, namely extends, between the two support bands 31 and 311 connecting them to each other.

In more detail, each transverse partition 6 has a central portion PC which is arranged between the support bands 31 and 311 to connect them and two peripheral portions PP opposite one another, each of which projects outwards from the respective support band 31 or 311, that is, from the opposite side of the support band 3 relative to the central portion PC. In other words, the two peripheral portions PP extend outwards from each support band 31 or 311.

The peripheral portions PP of the same transverse partition 6 are connected to each other so as to form a ring and fix the tubular shape of the main structure 2.

According to the example illustrated in FIGS. 3 and 6, the detection pads 5 have an annular (that is, three-dimensional) shape, therefore advantageously, the signal is acquired, in use, along an annular segment 17 that detects signals at 360° around the central axis X.

Advantageously according to the example illustrated in FIGS. 3 and 6, both the support bands 31 and 311 are inclined at an angle α relative to the central axis X. A development of the main structure 2 in plane is illustrated in FIG. 6. In FIG. 6, the inclination angle α can be seen relative to the central axis X. The width of the angle α is variable and determines the pitch of the helix in the three-dimensional main structure 2 and consequently affects the rigidity of the diagnostic device 1. Preferably, the inclination angle α of the support bands 3 ranges from 0° to 45°, in particular from 10° to 35°, more advantageously preferably from 15° to 30° relative to the central axis X.

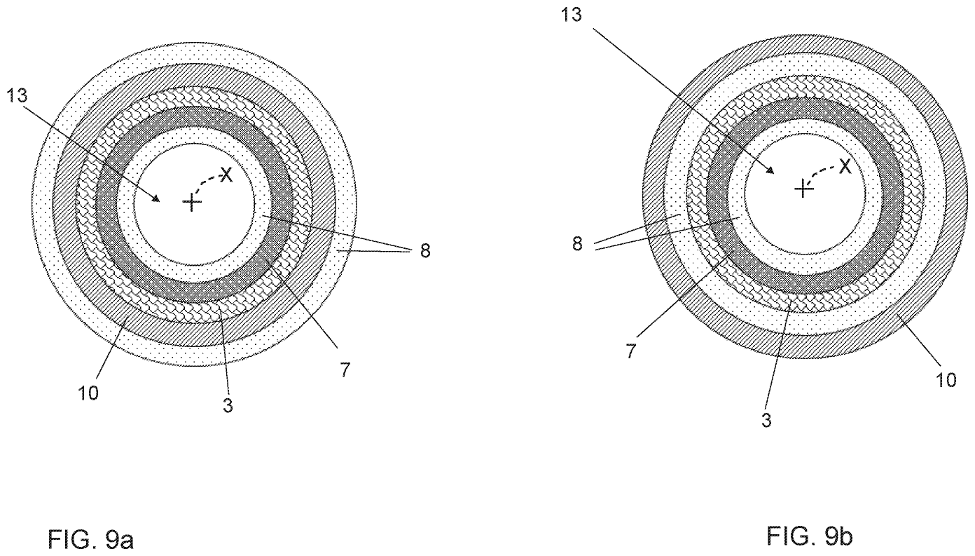

The diagnostic device 1 has an electrically conductive coating 7, an auxiliary matrix 8 and preferably a further electrically conductive coating 10 (FIGS. 2, 9a and 9b). In particular, the electrically conductive coatings 7 and 10 are two coatings that are different from one another in terms of material and/or function performed.

The electrically conductive coating 7 is applied on the detection pads 5 radially inwards (relative to the central axis X) of the main structure 2 and defines a sensitive element for detecting pressure (namely, for generating the signal proportional to the pressure). The electrically conductive coating 7 is preferably applied directly to the detection pads as indicated in FIGS. 9a and 9b. The electrically conductive coating 7 ensures the continuity of the signal between the detection pads 5 and the reading points of the signal in the area of the distal end ED of the main structure 2 (as illustrated in FIG. 6).

Advantageously, the electrically conductive coating 10 is applied radially more on the outside relative to the electrically conductive coating 7 and is configured to shield the diagnostic device 1 from the electromagnetic field generated by the human body. In other words, the electrically conductive coating 10 prevents the electromagnetic field from interfering with the detected signal and, therefore, from making an incorrect or altered acquisition of the signal, in particular pressure.

It should be noted that the terms radially “innermost” and “outermost” refer to the position along the radial direction (in particular, to the distance from the central axis X along the radial direction) in the diagnostic device 1, as illustrated in FIGS. 2-5, 7 and 9.

According to a possible variation illustrated in FIG. 9a, the electrically conductive coating 10 is arranged on the support bands 3 in the area of the opposite side to which the electrically conductive coating 7 is applied. In other words, in the diagnostic device 1 the electrically conductive coating is arranged on the radially outermost side (namely, the surface) of the support bands 3I and 3II; whereas the electrically conductive coating 7 is arranged on the radially innermost side (namely, the surface) of the support bands 3I and 3II.

Advantageously, the electrically conductive coating 10 is applied to the entire surface of the support band 3I and 3II. In other words, the electrically conductive coating 10 is applied as a continuous layer on the radially outermost surface of the main structure 2.

According to a variation illustrated in FIG. 9b, the electrically conductive coating 10 is arranged externally on the auxiliary matrix 8. In other words, in the diagnostic device 1 the electrically conductive coating 10 is arranged in the area of the radially outermost surface of the auxiliary matrix 8.

Advantageously, the electrically conductive coating 10 is applied to the entire surface of the auxiliary matrix 8. In other words, the electrically conductive coating 10 is applied as a continuous layer in the area of the radially outermost surface of the auxiliary matrix 8.

The auxiliary matrix 8 is configured to support, sustain and maintain the shape of the main structure 2 which is embedded therein. In particular, the auxiliary matrix 8 has mechanical and technological characteristics (for example, in terms of elastic modulus, that is, the so-called Young's modulus) such as to obtain the detected signal, in particular the pressure signal, which is comprised within a predefined range.

The auxiliary matrix 8 accommodates the structure 2 and isolates one detection pad 5 from the other (that is, one sensitive element from the other). In particular, the auxiliary matrix 8 wraps (in particular, incorporates at least partially, preferably completely) the main structure 2 and fills the gaps G (such as, for example, crevices or free cavities) between the support bands 3 to define the tubular body 9. The tubular body 9 (namely, the main structure 2 incorporated in the auxiliary matrix 8) substantially defines a hollow probe having a central through channel 13 within it. The central channel 13 enables introduction of any auxiliary equipment, such as, for example, a guide wire for introducing the catheter via endoscopic guidance and/or for administering drugs. As illustrated in FIG. 2, the central channel 13 is open in the area of the proximal end EP such as to cause any auxiliary equipment and/or drug administered to be released. The detection pads 5 are distributed along the central axis X and face the central channel 13.

As illustrated in FIGS. 3-5, the support bands 3 extend along the axis X and at least partially laterally delimit the central channel 13.

Advantageously, the diagnostic device 1 is of the disposable type, namely, single use. In other words, the diagnostic device 1 is not multi-use.

Advantageously, the main structure 2 is made of a plastic material, in particular of a film made of polyimide, and has a thickness ranging from 0.1 to 0.5 mm, in particular ranging from 0.2 to 0.3 mm. A possible material for creating the main structure 2 is, for example, the material marketed under the name Kapton®. Kapton® is usually marketed with a film of insulating material that is arranged on both sides (namely, the larger surfaces). It should be noted that in this case the electrically conductive coating 7 and the electrically conductive coating 10 are applied to opposite sides of the film of insulating material of Kapton®.

Advantageously, the electrically conductive coating 7 is made of silver, copper or materials with a high index of electrical conductivity.

Advantageously, the electrically conductive coating 10 is also made of silver, copper or materials with a high electrical conductivity index.

Alternatively, the electrically conductive coating 10 may be made of a material with conductive material particles dispersed therein, such as, for example, a silicone with carbon nanotube particles dispersed therein.

Advantageously, the auxiliary matrix 8 is made of an insulating material that has a dynamic viscosity ranging from 3 to 20 cP [with the abbreviation cP or cps we mean the unit of measurement “Centipoise” which is commonly used in the industry to indicate the dynamic viscosity of a material. According to the International System SI, 1 cP is equivalent to 1 mPas. Therefore, with reference to the International System, the auxiliary matrix 8 is made of an insulating material that has a dynamic viscosity ranging from 3 to 20 mPas] and a tensile strength from 200 to 550 psi [with the abbreviation psi we mean a unit of measurement, of the US customary system, of: pressure, stress, Young's modulus and maximum tensile strength. According to the International System SI, 1 psi is equivalent to approximately 0.006894757 N/mm². Therefore, with reference to the International System, the auxiliary matrix 8 is made of an insulating material having a tensile strength ranging from approximately 1.37895 to 3.79211 N/mm²]. For example, the auxiliary matrix 8 is made of a silicone which is different from that of the electrically conductive coating 10.

FIGS. 4a to 4c and 5a to 5c illustrate respective variations of the diagnostic device 1 according to the present invention. Hereinafter, in FIGS. 4a to 4c and FIGS. 5a to 5c, the components in common with the preferred embodiment of the diagnostic device 1 maintain the same numbering and are considered to be comprised therein without renaming them for the sake of brevity.

According to the example illustrated in FIGS. 4a to 4c, there are three support bands 3 3I, 3II and 3III, each of which extends helically, namely, is screw wound, along the central axis X defining the main netlike structure 2. In other words, the support bands 3I, 3II and 3III define the helix-shaped main structure 2. The detection pads 5 are obtained directly on each support band 3 with no interruption. Advantageously, in the example illustrated in FIGS. 4a to 4c, the detection pads 5 have a flat, substantially two-dimensional shape, which is circular or polygonal. Therefore, as illustrated in the cross-section of FIG. 4, the signal is acquired from the plurality of segments 17, in particular three, one for each support band 3. The segments 17 are distinct and separated from each other. According to the example illustrated in FIGS. 4a to 4c, the segments 17 are uniformly distributed around the central axis X.

According to a third and alternative embodiment, illustrated in FIGS. 5a-5c, there are at least two support bands 3, preferably more than two, and extend parallel side by side along the central axis X. In the example illustrated in FIGS. 5a to 5c, there are three support bands 3, 3I, 3II and 3III. The detection pads are obtained directly on each support band 3. In other words, the detection pads 5 are manufactured in one piece, with no interruption, with each support band 3. Advantageously, as illustrated in FIG. 5, in this embodiment the detection pads have a substantially two-dimensional circular or polygonal shape.

Therefore, as illustrated in the cross-section of FIG. 5c, the signal is acquired from the plurality of segments 17, in particular three, which are distinct and separate from each other. Preferably, the segments 17 are uniformly distributed around the central axis X.

Without loss of generality, all the support bands 3 of a diagnostic device 1 may:
have an opposite winding way around the central axis X (as in the examples illustrated in FIGS. 3a-3c and 4a-4c);
have a same winding way around the central axis X;
be parallel to each other (as in the example illustrated in FIGS. 5a-5c).

The method for the production of a diagnostic device 1 according to the present invention is described hereinafter.

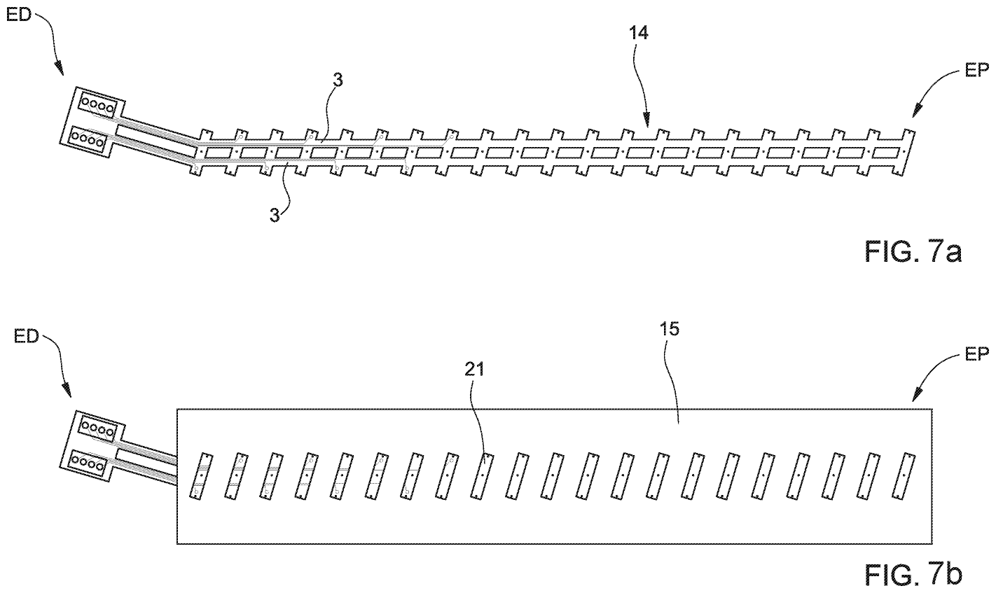

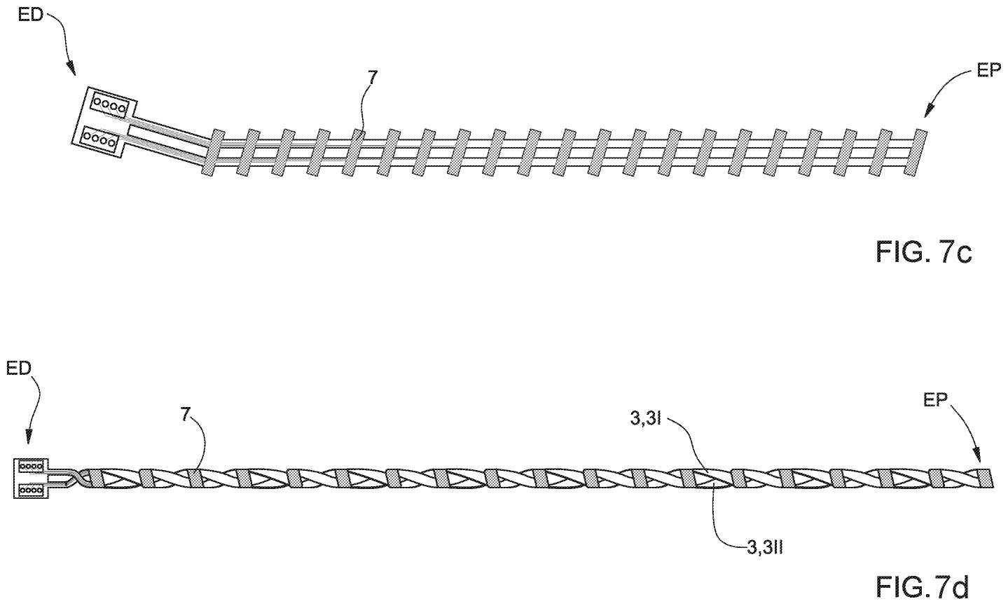

The method for the production of the diagnostic device 1 provides for a plurality of steps illustrated in FIGS. 7a-7d, which will be described more fully below.

The diagnostic device 1 is obtained starting from a blank 14 (illustrated in FIGS. 6 and 7a) made of insulating material, in particular polyimide, more specifically KAPTON®.

The method provides for a step of obtaining a blank 14 from a plastic material sheet (illustrated in FIG. 7a). The blank 14 having one or more support bands 3.

According to the preferred embodiment illustrated in FIGS. 6 and 7a, there are two support bands 3 and are inclined at an inclination angle α relative to the central axis X of the diagnostic device 1. Each support band 3 is provided with electrical tracks 4 and with a respective plurality of detection pads 5 spaced apart from one another and configured to generate, in use, a signal, in particular a pressure signal.

Preferably, the step of obtaining a blank 14 comprises a previous sub-step in which the blank 14 is obtained from a plastic material sheet, in particular made of polyimide or KAPTON®, by cutting (for example, by laser cutting) or punching.

The method also has a step of providing one or more sensitive elements 5, in particular detection pads 5, for each band 3. Each sensitive element 5 is configured to acquire signals, in particular pressure signals. For each band 3, electrical tracks 4 are connected to the sensitive elements 5 (that is, to the detection pads 5) for the transmission of signals.

Next, the method provides for a step of applying an electrically conductive coating 7.

The application step provides for applying the electrically conductive coating 7 on the blank 14 in the area of the detection pads 5.

Advantageously, as illustrated in FIG. 7b, the application step comprises the sub-step of arranging a mask 15 on the blank 14. The mask 15 is provided with a plurality of openings 21 which are configured to overlap the detection pads 5. In this way, with the help of the mask 15, the electrically conductive coating 7 is accurately applied only in the desired areas (namely, the detection pads 5), as illustrated in FIG. 7c; whereas it is not applied on the remaining parts of the structure 2.

Advantageously, in a different sub-step of application, the electrically conductive coating 7 is also applied in the area of the inner surface of the central channel 13, such as to make it electrically conductive and therefore allow it to act, in use, as a ground electrode.

Alternatively, an electrical ground wire, namely an earth wire, may be arranged on the blank 14. The electrical ground wire may be arranged on the blank 14 after the step of applying the electrically conductive coating 7, or it may be introduced at a later step.

Next, as illustrated in FIG. 7d, the method provides for a step of creating the cylindrical main structure 2 with the blank 14, in particular by rolling the blank 14. In other words, in this step the main structure 2 is obtained from the blank 14, by keeping the electrically conductive coating 7 radially on the inside (namely, on the inner surface) of the cylindrical main structure 2.

In particular, for the implementation of the embodiments illustrated in FIGS. 3 and 4, the step of creating the main structure 2 provides for the sub-step of rolling the blank 14 so as to obtain the helix-shaped main structure 2, in which each support band 3 extends helically around the central axis X. In particular, during the sub-step of rolling, the blank 14 is rolled and the detection pads 5, that is, the electrically conductive coating 7, are kept facing inwards.

The main three-dimensional structure 2 is formed through rolling, which is therefore defined by a single element, without the need for further assembly operations.

In accordance with FIGS. 2 and 3, by rolling the blank 14, the detection pads 5 acquire the final, namely annular, shape and are connected to each other by the support bands 3 (which are insulating) which are arranged to form the helix-shaped main structure 2.

In particular, for creating the main structure 2 in accordance with the alternative illustrated in FIGS. 5a-5c, the blank 14 (which is different from the blank 14 illustrated in FIG. 6) is arranged so that the support bands 3 are arranged parallel around the central axis X, so as to form the cylindrical main structure 2.

Advantageously, once the main structure 2 has been obtained from the blank 14, a sub-step of connection, preferably by means of welding, of the respective peripheral portions PP of the same transverse partition 6 is carried out in order to define each detection pad 5.

Finally, a step is envisaged to create the auxiliary matrix 8, thus incorporating the main structure 2, fill the gaps G between the support bands 3 and form, namely obtain, the tubular body 9.

Advantageously, the auxiliary matrix 8 is created by casting, namely injection, of the material into a mould so that the main structure 2 is embedded within it.

Advantageously, the method comprises a step of applying the electrically conductive coating 10.

If the electrically conductive coating 10 is applied directly on the main structure 2 (as illustrated in FIG. 9a), then this application step takes place upstream of the step of creating the main structure 2. In particular, the method comprises a sub-step of applying the electrically conductive coating 10 on the blank 14 in the area of the side (namely, surface) opposite to the application of the electrically conductive coating 7. The step of applying the electrically conductive coating 10 on the blank 14 may take place upstream or downstream of the step of applying the electrically conductive coating 7.

Advantageously, if the electrically conductive coating 10 is applied on the auxiliary matrix 8 (as in the variation illustrated in FIG. 9b), then this application takes place downstream of the step of creating the auxiliary matrix 8.

In use, the proximal end EP of the diagnostic device 1 is connected to the control unit 110; whereas the distal end ED is inserted into the patient's orifice. Once the diagnostic device 1 is positioned within the patient's cavity (for example, the esophagus), the device 1 undergoes radial deformations due to the pressure wave during swallowing.

Signals proportional to the perceived pressure and relative to the specific application are generated by means of the detection pads 5.

In particular, the signals are generated according to the following mathematical relationship:

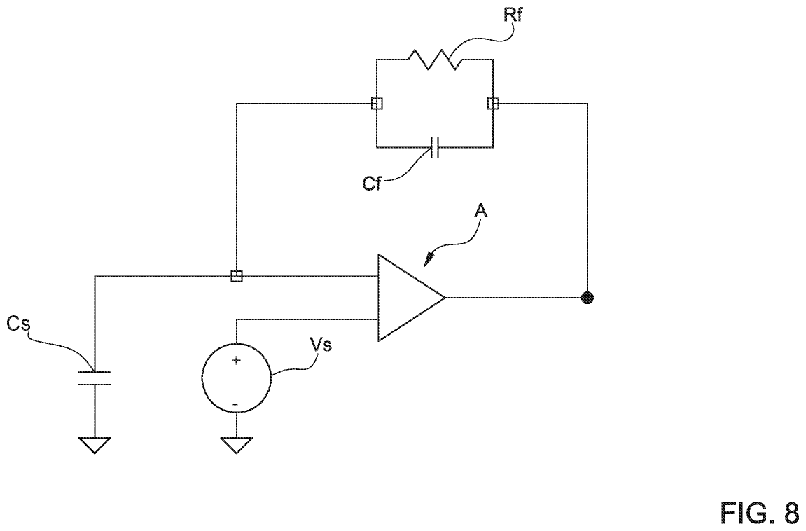

C=ε*A/d
Wherein:
C is the electrical capacity of a capacitor (in particular, of the capacitor Cs of FIG. 8);
ε is the permittivity of the dielectric;
A is the surface of the electrically conductive coating 7 applied on the detection pads 5 [first plate of the capacitive (namely, of the capacitor Cs)] and on the central channel 13 [second plate of the capacitive (namely, of the capacitor Cs)]; and d is the distance between the two plates.

These signals are exchanged with the control unit 100, which acquires the data required for the prescribed examination (such as, for example, intra-esophageal pressure, while performing an established diagnostic protocol) according to a certain logic.

In particular, as illustrated in FIG. 8, in the area of each detection pad 5 the signal (the pressure signal) is acquired by exploiting capacitive technology. Therefore, the application of a stimulus, for example a pressure stimulus, on a section of the diagnostic device 1 causes reduction of the distance relative to the central ground electrode leading to a change in the measured capacitance. In particular, as already indicated previously, the electrically conductive coating 7 arranged on the radially inner surface of the detection pads 5 represents one of the capacitive plates, whereas the corresponding section of central channel 13, also coated by the electrically conductive coating 7, represents the other capacitive plate.

The capacitive technology provides for acquisition of the signal by means of a voltage generator Vs arranged in parallel to the capacitor Cs, after which the acquired signal is sent to a signal amplifier A and then the signal is conditioned in retraction with the help of a resistor Rf and a capacitor Cf arranged in parallel. Furthermore, there is an envelope detector (not shown) which is arranged upstream of the signal conversion from analogue-to-digital to reduce the sampling frequency.

Alternatively, other detection technologies, other than capacitive technology, may also be applied.

The diagnostic device 1 and the production method subject of the present invention have a number of advantages.

Firstly, thanks to the netlike main structure 2, the diagnostic device 1 is equally flexible and deformable in any direction orthogonal to the central axis X. Therefore, with the diagnostic device 1 it is no longer necessary to perform unpleasant twists of the diagnostic device 1 in order to bend it during introduction into the patient's cavity, with obvious advantages for the patient's well-being.

Secondly, the diagnostic device 1 is quick and inexpensive to manufacture, which allows it to be used as a single-use diagnostic device 1. Therefore, after each use, the diagnostic device 1 does not have to be cleaned and disinfected; instead, it can be disposed of.

Furthermore, the diagnostic device 1 enables reliable pressure measurements to be carried out.

Advantageously, the presence of the central channel 13 makes it possible to introduce (through the central channel 13) auxiliary devices and/or the administration of drugs topically into the diagnostic device 1. Therefore, the diagnostic device 1 also has a therapeutic function in addition to the diagnostic function.

Advantageously, the central channel 13 allows the introduction of an endoscopic guide to ease the opening of the patient's cavity and the insertion of the diagnostic device 1.

The central channel 13 also provides greater axial rigidity, thus increasing the compressive strength to which the diagnostic device 1 is subjected during insertion.

The auxiliary matrix 8 which wraps the main structure 2 provides mechanical support and protection to the main structure 2. Furthermore, the auxiliary matrix 8 allows the relative arrangement of the support bands 3 to be maintained and therefore to maintain the shape of the main structure 2.

By using the diagnostic device 1 with capacitive technology, it is possible to keep manufacturing costs low while achieving high performance.

The diagnostic device 1 subject of the present invention, with the same diameter and length as known devices, allows to have a spatial resolution at the maximum of clinical relevance.

The method for manufacturing the diagnostic device 1 is simple and also has low production costs.

In addition, by using an internal main structure 2 like the one illustrated in FIG. 3, it is possible to have the acquisition of the signal from the continuous annular segment 17 and affecting the entire cross-section of the diagnostic device 1, in this way it is possible to advantageously obtain the signal on the entire cylindrical portion of the respective cavity.

Finally, by manufacturing the diagnostic device 1 with the electrically conductive coating 10, it is possible to obtain a more accurate signal acquisition, as there is no interference with the electromagnetic field generated by the human body. In fact, as the electrically conductive coating 10 is applied radially more on the outside relative to the electrically conductive coating 7, it acts as a shield.

1. A method for the production of a diagnostic device, for endoscopic examinations, comprising the steps of:
obtaining a blank from a plastic material sheet; the blank comprising one or more support bands provided with electrical tracks;
providing a plurality of sensitive elements uniformly distributed along each band; wherein each sensitive element is configured to generate signals, and is connected to the electrical tracks for the transmission of signals;
applying a first electrically conductive coating on the blank in the area of the sensitive elements;
creating a main structure from the blank, keeping the first electrically conductive coating radially on the inside of the main structure; and
creating an auxiliary matrix (8), which wraps the main structure and fills the gaps between the support bands, so as to obtain a tubular body having a central through channel which is at least partially laterally delimited by said support bands; wherein said sensitive elements are distributed along the central axis and face the inside of the central channel; wherein the main structure is helix-shaped, that is, each support band extends helically around the central axis so that the main structure is bendable relative to a plurality of deformation axes with different directions to each other.

2. The method according to claim 1, wherein the blank comprises two support bands and sensitive elements, which are respectively defined by a partition provided with a central portion connecting the two support bands and two peripheral portions extending outwards from each support band; wherein the method comprises the step of connecting the peripheral portions of the same transverse partition in order to define each sensitive element.

3. The method according to claim 1, wherein, during the step of creation of the main structure, the blank is rolled so as to obtain a helix-shaped main structure, in which each support band extends around the central axis in a helical manner.

4. The method according to claim 1, wherein, during the step of creation of the main structure, the blank is arranged so that the support bands are arranged in a parallel manner around the central axis.

5. The method according to claim 1, comprising a further step of applying a second electrically conductive coating; wherein this step takes place upstream of the step of creation of the main structure or downstream of the step of creation of the auxiliary matrix.

6. A diagnostic device for endoscopic examinations, configured to be inserted into an orifice of the body of a patient; the diagnostic device comprising a main structure, which has a tubular shape along a central axis and comprises, in turn, two or more support bands, each provided with at least one or more electrical tracks and with a plurality of sensitive elements distributed along each support band;
wherein the sensitive elements are spaced apart from one another and each of which is configured to generate, in use, signals;
wherein the main structure has a central through channel which is at least partially laterally delimited by said support bands;
wherein said sensitive elements are distributed along the central axis and face the inside of the central channel; and
wherein the main structure is helix-shaped, that is, each support band extends helically around the central axis so that the main structure is bendable relative to a plurality of deformation axes with different directions.

7. The diagnostic device according to claim 6, wherein the main structure comprises a first electrically conductive coating, which is applied on the sensitive elements radially towards the inside of the main structure and defines an electrical contact with the outside; and an auxiliary matrix, which wraps the main structure and fills the gaps between the support bands, so as to obtain a tubular body.

8. The diagnostic device according to claim 7, comprising a second electrically conductive coating (10), which is applied radially more on the outside relative to the first electrically conductive coating; wherein the second electrically conductive coating is applied to the main structure in the area of the opposite side relative to the side of application of the first electrically conductive coating or is arranged on the radially outer surface of the auxiliary matrix.

9. The diagnostic device according to claim 6, wherein the support bands are inclined, relative to the axis, by an inclination angle ranging from 0° to 45°.

10. The diagnostic device according to claim 6, wherein there are two support bands, each of which extends along the central axis in a helical manner, thus defining the main structure with a netlike shape, and the sensitive elements are obtained directly on each support band with no interruptions.

11. The diagnostic device according to claim 10, wherein each sensitive element is defined by a transverse partition, which extends between the two support bands; the partition has a central portion, which is arranged between the support bands and a peripheral portion, which projects outwards, on the opposite side relative to the central portion, from each support band; the peripheral portions of the same partition are joined to one another, thus forming a ring.

12. The diagnostic device according to claim 6, wherein the main structure is made of a plastic material, and has a thickness ranging from 0.1 to 0.5 mm.

13. The diagnostic device according to claim 6, wherein the coating is made of an insulating material with a viscosity ranging from 3 to 20 cps (from 3 to 20 mPas), and a tensile strength ranging from 200 to 550 psi (from 1.37895 to 3.79211 N/mm²).

14. A diagnostic system comprising a diagnostic device obtained according to claim 6 and a control unit.
